(19) United States
(12) Patent Application Publication
(10) Pub. No.: US 2025/0249228 A1
(43) Pub. Date: Aug. 7, 2025

(54) FLUID CONNECTOR ASSEMBLY

(71) Applicant: CareFusion 303, Inc., San Diego, CA (US)

(72) Inventors: Jason Andrew WINE, Brea, CA (US); Ryan Callahan, Long Beach, CA (US); Leah Paige Gaffney, Orange, CA (US)

(21) Appl. No.: 18/429,779

(22) Filed: Feb. 1, 2024

(51) Int. Cl. A61M 39/10

(52) U.S. Cl. CPC A61M 39/10; A61M 2039/1016; A61M 2039/1027; A61M 2039/1077; A61M 2205/0216

(57) ABSTRACT

Fluid connector assemblies that seal off fluid paths in the respective connectors are disclosed. The connectors may be coupled via a connecting mechanism that that provides a threshold retention force. When an external force greater than the threshold force is applied to the fluid connector assembly, the snap mechanism may no longer maintain the connectors together, causing the connectors to decouple from each other. Once decoupled, the connectors may be sanitized and/or replaced before being recoupled.

### TECHNICAL FIELD

The present disclosure relates generally to medical fluid connectors and, more particularly, to a fluid connector assembly that includes medical connectors that decouple with each other due to an applied force, with each medical connector designed to automatically seal off their respective fluid paths. The decoupling may be due to intentional or unintentional separation between the medical connectors.

### BACKGROUND

The present disclosure relates generally to medical fluid connectors and, more particularly, to coupling devices for connecting and disconnecting medical tubing.

Medical connections are widely used in fluid delivery systems such as those used in connection with intravenous (IV) fluid lines, blood access, hemodialysis, peritoneal dialysis, enteral feeding, drug vial access, and other procedures.

In some instances, the medical connection can become dislodged or disconnected in an unintended manner. For example, medical tubing of an IV set that is coupled to a catheter can become dislodged when an unintended or unexpected forces is exerted upon the catheter, which may exceed the design limitations of the catheter securement method. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

### SUMMARY

In accordance with at least some embodiments disclosed herein is the realization that unintended dislodgement or disconnection of a medical connection, such as a medical fluid line, can result in injury to a patient or a medical professional, such as by depriving the patient of a medicament, increasing the potential for infection to the patient, and exposing the medical professional to medicaments.

Aspects of the present disclosure provide fluid connector assemblies with medical connectors, each of which include one or more fluid paths, that respond to unintentional or unexpected external forces by decoupling from each other and sealing off their respective fluid paths. The decoupling may include automatic decoupling using bellows or other elastically compressible member that decompress and return to their original shape external forces are no longer acting upon them. Beneficially, fluid connector assemblies described herein can limit or prevent patient blood loss, IV fluid loss, infection, and medical delivery delays. Further, aspects of the present disclosure provide connecting mechanisms that aid in the connection of the fluid connector assembly in the event that the fluid connector assembly is decoupled.

According to certain embodiments, a fluid connector may include a first connector having a first housing, a second connector having a second housing. The second connector may be configured to couple with the first connector. The fluid connector may further include a connecting mechanism disposed on at least one of the first connector and the second connector for removably coupling the first connector and the second connector. When a predetermined pull force is applied to at least one of the first connector and the second connector, the connecting mechanism disengages thereby allowing the first connector and the second connector to be decoupled. When the first connector and the second connector are decoupled, an interior surface of one of the first connector is exposed thereby allowing for disinfection.

In some embodiments, the first connecting mechanism includes a first connecting member coupled to one of the first connector or the second connector. The first connecting member may include a projection member disposed at a first end of the first connecting member.

In some embodiments, the fluid connector includes a second connecting member. The first connecting member may be coupled to the first connector and the second connecting member is coupled to the second connector. The second connecting member may include a groove for receiving the projection member. When the first connector and the second connector are coupled, the first connecting member and the second connecting member may be coupled such that the projection is received within the groove. When the predetermined force is applied, the first connecting member and the second connecting member may be decoupled.

In some embodiments, the first connecting member is a press ring. The first connecting member may be flexible. The second connecting member may further include a groove for receiving the projection of the press ring.

In some embodiments, when the and the second connector are coupled, the first connecting member and the second connecting member are coupled and, when the predetermined force is applied, the first connecting member and the second connecting member may be decoupled.

In some embodiments, the projection is configured to engage an elastomer ring and, when the predetermined force is applied, the projection may disengage the elastomer ring thereby decoupling the first connector and the second connector.

In some embodiments, the connecting member includes an elastic band and one or more threads disposed around the first connector or second connector. The elastic band may be disposed around the threads. The elastic band may be configured to deflect until the predetermined threshold force is applied. When the predetermined threshold force is applied, the elastic band may expand allowing the threads to pull thereby decoupling the first connector and the second connector.

According to certain embodiments, a fluid connector may include a first connector having a first housing, a second connector having a second housing. The second connector may be configured to couple with the first connector. The fluid connector may further include a connecting mechanism disposed on at least one of the first connector and the second connector for removably coupling the first connector and the second connector. When a predetermined pull force is applied to at least one of the first connector and the second connector the first and second connectors may be decoupled.

In some embodiments, the connecting mechanism includes a first connecting member coupled to one of the first connector or the second connector. The first connecting member may include a projection member disposed at a first end of the first connecting member.

In some embodiments, the fluid connector includes a second connecting member. The first connecting member may be coupled to the first connector and the second connecting member may be coupled to the second connector. The second connecting member may include a groove for receiving the projection member. When the first connector and the second connector are coupled, the first connecting member and the second connecting member may be coupled such that the projection is received within the groove, and, when the predetermined force is applied, the first connecting member and the second connecting member may be decoupled.

In some embodiments, when the first connector and the second connector are coupled, the first connecting member and the second connecting member may be coupled, and, when the predetermined force is applied, the first connecting member and the second connecting member may be decoupled.

In some embodiments, the projection may be configured to engage an elastomer ring and, when the predetermined force is applied, the projection disengage the elastomer ring thereby decoupling the first connector and the second connector.

According to certain embodiments, a fluid connector may include a fluid connector may include a first connector having a first housing, a second connector having a second housing. The second connector may be configured to couple with the first connector and may include a first connector member and a second connector member. The fluid connector may further include a connecting mechanism disposed on at least one of the first connector and the second connector for removably coupling the first connector and the second connector thereby allowing the first connector member to be decoupled form the second connector member.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a coupling device for medical tubing in use with an IV set coupled to a patient, in accordance with aspects of the present disclosure.

FIG. 2 illustrates a perspective view of an embodiment of a coupling device for medical tubing, in accordance with aspects of the present disclosure.

FIG. 3 illustrates a perspective view of the coupling device for medical tubing of FIG. 2, in accordance with aspects of the present disclosure.

FIG. 4 illustrates a cross-sectional view of the coupling device for medical tubing of FIG. 2, in accordance with aspects of the present disclosure.

FIG. 5 illustrates a cross-sectional view of the coupling device for medical tubing of FIG. 2 and a mating connector, in accordance with aspects of the present disclosure.

FIG. 6 illustrates a cross-sectional detail view of the coupling device for medical tubing of FIG. 4, in accordance with aspects of the present disclosure.

FIG. 7 illustrates a cross-sectional detail view of an embodiment of a coupling device for medical tubing, in accordance with aspects of the present disclosure.

FIGS. 8-10 illustrate cross-sectional views of the coupling device for medical tubing of FIG. 2 and a mating connector, in accordance with aspects of the present disclosure.

FIG. 11 illustrates a cross sectional view of an embodiment of a connecting mechanism for the coupling device, in accordance with aspects of the present disclosure.

FIG. 12 illustrates a cross sectional view of an embodiment of a connecting mechanism for the coupling device, in accordance with aspects of the present disclosure.

FIG. 13 illustrates a cross sectional view of an embodiment of a connecting mechanism for the coupling device, in accordance with aspects of the present disclosure.

FIG. 14 illustrates a cross sectional view of an embodiment of a connecting mechanism for the coupling device, in accordance with aspects of the present disclosure.

FIG. 15 illustrates a cross sectional view of an embodiment of a connecting mechanism for the coupling device, in accordance with aspects of the present disclosure.

FIG. 16 is a perspective view of an extension set including a connector assembly, in accordance with some embodiments of the present disclosure.

FIG. 17 illustrates a cross-sectional view of a connector assembly coupled with a mating needleless connector, in accordance with some embodiments of the present disclosure.

FIG. 18 illustrates a partial perspective view of the connector assembly of FIG. 17, in accordance with some embodiments of the present disclosure.

FIG. 19 illustrates an operational view of the connector assembly decoupled from the mating needleless connector due to pivoting open of a distal connector of the connector assembly when the connector assembly is subject to a proximal-direction force above a predetermined threshold, in accordance with some embodiments of the present disclosure.

FIG. 20 illustrates a cross-sectional operational view of a proximal connector of the connector assembly decoupled from the connector assembly for swabbing, in accordance with some embodiments of the present disclosure.

FIG. 21 illustrates a cross-sectional operational view of the proximal connector of the connector assembly coupled to the rest of the connector assembly after swabbing, in accordance with some embodiments of the present disclosure.

FIG. 22 is an elevation view of a detachable connector assembly, in accordance with some embodiments of the present disclosure.

FIG. 23 illustrates a cross-sectional view of the connector assembly of FIG. 22, in accordance with some embodiments of the present disclosure.

FIG. 24 is a detail view of the connector assembly of FIG. 23, in accordance with some embodiments of the present disclosure.

FIG. 25 is a cross-sectional view of an arm for a connector assembly, in accordance with some embodiments of the present disclosure.

FIG. 26 is a cross-sectional view of the arm of FIG. 25, in accordance with some embodiments of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a coupling device for medical tubing. The coupling device for medical tubing can provide for efficient and safe maintenance of fluid connections, such as the connections used for transferring medical fluids toward or away from a patient. The coupling device can maintain the fluid connection of a medical tubing by resisting disconnection when a pulling or tension force is applied to the coupling device, such as when a patient moves or when the medical tubing is pulled away from the patient. The coupling device also prevents injury to a patient or a caregiver by permitting disconnection of the fluid connection when a pulling or tension force exceeds a threshold. The coupling device also provides for efficient and safe reconnection of the fluid line by permitting the coupling device to be reconnected to the fluid transfer device, such as a catheter or needleless connector, thereby reestablishing the fluid pathway without requiring replacement of the coupling device. The coupling device can also include features to permit safe and efficient sterilization and cleaning for portions of the device before connection and reconnection therebetween.

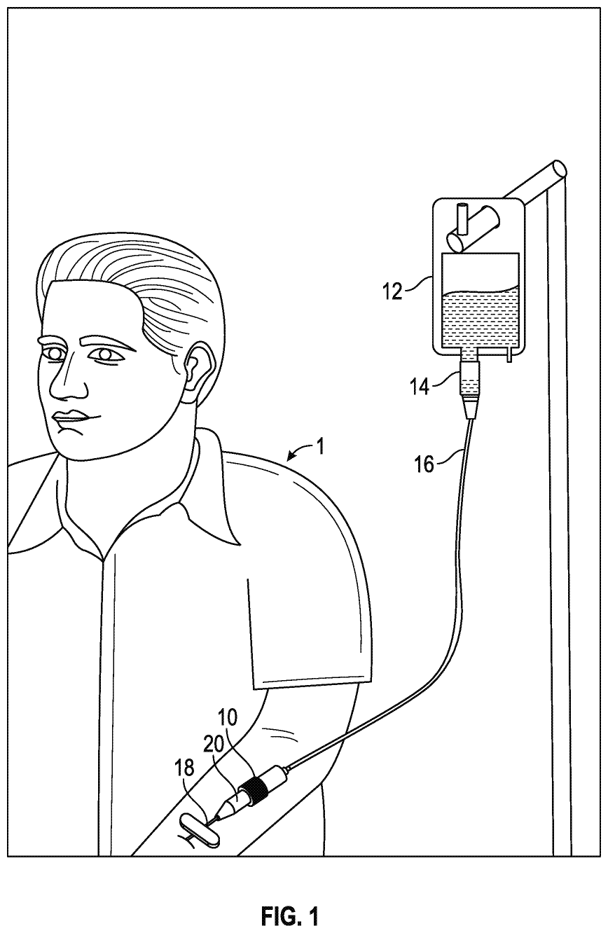

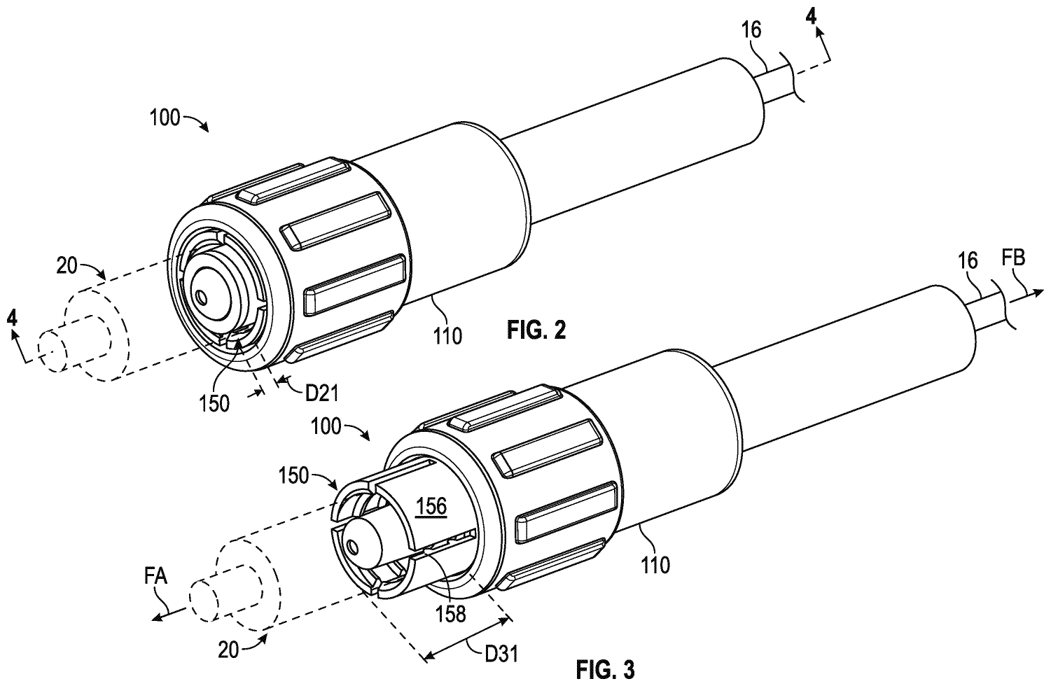

Referring now to the figures, FIG. 1 illustrates an embodiment of a coupling device for medical tubing in use in accordance with aspects of the present disclosure. The coupling device 10 is coupled with tubing of an IV set, which is being used to direct a fluid to a patient 1. The IV set can include a medicament bag 12, a drip chamber 14, tubing 16, and an IV catheter 18.

The coupling device 10 fluidly connects the tubing 16 to the IV catheter 18. Although the coupling device 10 is illustrated being coupled along a fluid pathway of an IV set, between a medicament bag 12 and a patient 1, it should be understood that the coupling device 10 can be connected within other fluid pathways, such as between a patient and an IV pump or between a patient and a dialysis machine. The coupling device 10 can also be connected along another portion of a fluid pathway. For example, the coupling device 10 can be connected along a proximal portion of the fluid pathway, such as being connected between the tubing 16 and the medicament bag 12 or other fluid therapy device.

The coupling device 100 includes a housing 110 and an inner body 150 positioned, at least partially, within an inner cavity of the housing 110. The coupling device may further include a connecting mechanism 200, 300, 400, 500, 600 configured to couple with the mating connector 20. The structure of the connecting mechanism 200, 300, 400, 500, 600 will be described in further detail below.

When the coupling device 100 is in the first configuration, the inner body 150 is in a first position within the cavity of the housing 110, whereby the coupling sleeve of the inner body 150 resists separation or disconnection of the mating connector 20 from the coupling device 100.

In some embodiments of the present disclosure, when the inner body 150 is in the first position, an end of the inner body is approximately aligned or coplanar with an end of the housing. In some embodiments, a portion of the connecting mechanism 200, 300, 400, 500, 600 or another portion of the inner body 150 may extend through the end of the housing by a first distance D21 when the inner body 150 is in the first position.

When the coupling device 100 is in a first configuration, with the inner body 150 in a first position, the coupling device 100 may be able to resist unintended separation of the mating connector 20 from the coupling device 100 by resisting biasing or flexing of the connecting mechanism 200, 300, 400, 500, 600 away from mating connector 20. In some embodiments of the present disclosure, biasing of the connecting mechanism 200, 300, 400, 500, 600 radially outward is resisted by the housing 110, such that, when the mating connector 20 and the coupling device 100 are pulled away from each other, the connecting mechanism 200, 300, 400, 500, 600 will engage against a portion of the housing 110, thereby resisting movement of the mating connector 20 out of the sleeve.

The coupling device 100 can be moved from the first configuration to the second configuration, which is illustrated in FIG. 3, when a pulling or tension force exceeds a threshold of the coupling device 100. For example, when a patient moves in an unintended manner, or when the medical tubing is pulled away from the patient, a pulling or tension force FA, FB may be applied to the mating connector 20 or the tubing 16 in a direction away from the coupling device 100. In at least some instances of the present disclosure, a pulling or tension force FA is in a distal direction relative to the coupling device, and a pulling or tension force FB is in a proximal direction.

If the pulling or tension force FA, FB exceeds a first threshold of the coupling device 100, any of the housing 110 and the inner body 150 can move away from each other so that the inner body 150 moves from the first position to a second position. The movement of the housing 110 and the inner body 150, relative to each other, can be along a longitudinal axis of the inner body 150 that extends between the mating connector 20 and an end portion of the tubing 16 connected to the coupling device 100.

In the second position of the inner body 150, at least a portion may extend out of the cavity and away from an end of the housing 110 by a second distance D31. The second distance may be greater than the first distance between the distal end of the sleeve and the end of the housing when the inner body 150 is in the first position.

If a pulling or tension force FA, FB exceeds a second threshold of the coupling device 100, the connecting mechanism 200, 300, 400, 500, 600 may be configured such that the engagement of the mating connector 20 against the sleeve can cause at least a portion of the connecting mechanism 200, 300, 400, 500, 600 to bias or flex away from the mating connector 20. When the connecting mechanism flexes away from the mating connector 20, the resistance to separation of the coupling device 100 from the mating connector 20 may be reduced, relative to when the coupling device 100 is in the first configuration, thereby permitting the coupling device 100 to separate from the mating connector 20. In some embodiments, engagement of a thread of the mating connector 20 against a thread of the inner body 150 when the force FA, FB exceeds the second threshold causes the portion of the sleeve to flex away from the mating connector 20. In some aspects of the present disclosure, a thread of the coupling device 100 can skip over or pass a thread of the mating connector 20 when the force FA, FB exceeds a second threshold of the coupling device 100.

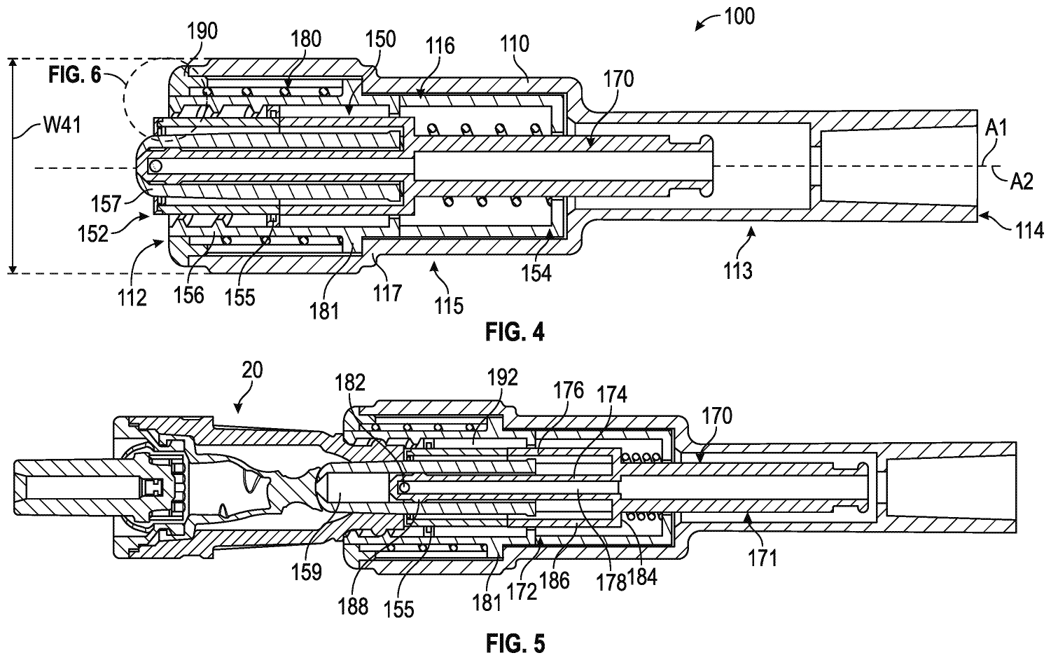

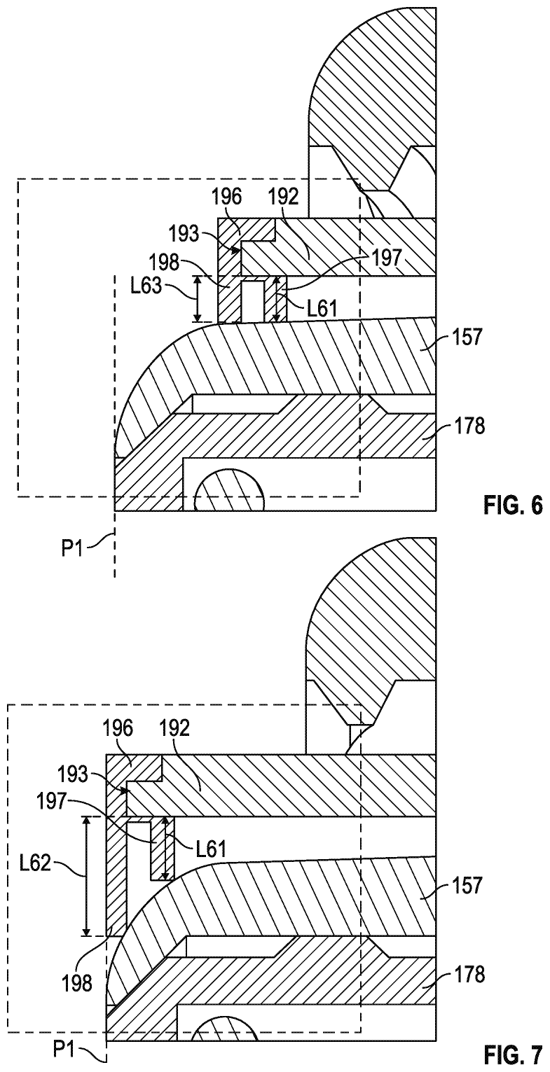

FIG. 4 illustrates a cross-sectional elevation view of the coupling device 100. The coupling device 100 may include the housing 110, the inner body 150, and an inner body biasing element 180. The inner body 150 may be configured to resist and/or permit separation of a mating connector 20 from the coupling device. In some aspects of the present disclosure, separation of a mating connector 20 from the coupling device may be resisted when the inner body is in a first position, and separation of a mating connector 20 from the coupling device may be permitted when the inner body is in a second position. The first position of the inner body 150 is shown, for example, in FIGS. 4 and 5, and the second position of the inner body 150 is shown, for example, in FIGS. 6 and 7.

The inner body 150 may be located within a cavity formed by the inner surface of the housing 110 and may be movable between the first and second positions. The inner body 150 can be positioned within a location of the cavity along a distal end portion of the housing. An inner body biasing element 180 can also be positioned within the cavity to bias the inner body 150 in a proximal direction to resist separation of a mating connector 20 from the coupling device 100.

The housing 110 may include a proximal end portion 113 forming a first end 114, a distal end portion 115 forming a second end 112. The first end 114 is opposite to the second end 112, and a longitudinal axis A1 is defined between the first and second ends of the housing. An inner surface 116 of the housing forms a cavity 118 that extends between the first end 114 and the second end 112, and is configured to receive the inner body 150 therein.

The first end 114 of the housing, or a portion of the housing adjacent to the first end 114, may form a first opening of the housing which may be configured permit tubing to be positioned through the first opening into the cavity. The second end 112 of the housing, or a portion of the housing adjacent to the second end 112, may form a second opening of the housing which may be configured to permit the inner body 150 to be inserted through the second opening into the cavity.

In some embodiments of the present disclosure, the inner surface 116 of the housing forms a ridge 117 that extends between the first end 114 and the second end 112 of the housing. In some aspects of the present disclosure, a portion of the inner body 150 is positioned between the ridge 117 and the second end 112 of the housing, and the inner body 150 is longitudinally movable between the ridge 117 and the second end 112 of the housing.

In embodiments of the present disclosure, the inner surface 116 of the housing forms any of a groove and/or ridge, and the inner body 150 forms the other of a groove and/or ridge, where the groove and/or ridge of the housing and inner body can extend in a longitudinal direction that is approximately parallel to a longitudinal axis A1 of the housing.

The groove and/or ridge of the housing 110 may be configured to mate with or engage against the inner body 150 such that, when the housing 110 is rotated around the longitudinal axis A1, the portion of the housing 110 forming the groove and/or ridge can engage against the portion of the inner body 150, thereby rotating the inner body 150 with the housing 110. By rotationally coupling the housing 110 and the inner body 150, the housing 110 can be rotated to engage threads of the inner body 150 with complementary threads of a mating connector 20.

The second end portion 152 of the inner body may also form a luer protrusion 157 configured to engage against a mating connector 20 and form a portion of a fluid passageway through the coupling device 100.

The luer protrusion 157 may be formed by the second end portion 152 of the inner body and may have an outer surface forming a male luer shape. The structure and orientation of the luer protrusion 157 may be such that the luer protrusion extends through the recess defined by the coupling sleeve 156 which extends around a perimeter or circumference of the luer protrusion 157.

In some embodiments of the present disclosure, an inner surface of the coupling sleeve 156 is spaced apart from the outer surface of the luer protrusion 157. The space between the coupling sleeve 156 and the luer protrusion 157 can permit a mating connector 20 to be affixed to the coupling device 100 with a portion of the mating connector 20 positioned between the coupling sleeve 156 and the luer protrusion 157. In some aspects of the present disclosure, the luer protrusion 157 is configured to displace a valve of the mating connector 20.

The inner surface of the luer protrusion may form a portion of the valve passage 159 through the inner body 150 and along the second end portion 152 thereof. The valve passage 159 can permit a fluid to through the coupling device 100 when coupled with a mating connector 20 in any of the first and second positions of the inner body 150.

An inner body biasing element 180 may be positioned in the cavity of the housing 110 to direct or bias the inner body 150 toward the first position. To bias the inner body 150 toward the first position, or in a direction toward the first end 114 of the housing, the biasing element 180 can be positioned between the inner body 150 and the second end 112 of the housing.

The inner body biasing element 180 can be any of a spring, a resilient bellows, or any similar structure configured to move the inner body 150. A first end of the inner body biasing element 180 can engage against the inner body 150, and a second end of the inner body biasing element 180 can engage against a portion of the housing 110 at the second end 112 thereof.

In some embodiments of the present disclosure, a portion of the inner body 150 forms a flange 181 configured to engage against the inner body biasing element 180. The flange 181 may be positioned along the second end portion 152 of the inner body and extend radially outward in a direction away from the longitudinal axis A2 of the inner body. The flange 181 can extend around the perimeter of the inner body 150 or can be formed by a series of discontinuous flanges 181 that are separated from each other around the perimeter of the inner body 150.

When the inner body 150 is positioned within the cavity of the housing 110, a first end of the inner body biasing element 180 can engage against the flange 181, and a second end of the inner body biasing element 180 can engage against the housing 110. The biasing element 180 exerts a force on the flange 181 of the inner body, thereby biasing the inner body 150 in the proximal direction toward the first end 114 of the housing.

In some embodiments of the present disclosure, the ridge 117 of the housing is configured to resist movement of the inner body 150 toward the first end 114 of the housing. When the inner body 150 is in the first position, the flange 181 of the inner body can engage against the ridge 117 of the housing flange 120, and when the inner body 150 is in the second position, the flange 181 of the inner body is spaced apart from the ridge 117 of the housing.

In some embodiments of the present disclosure, a cap 190 is coupled to the second end 112 of the housing to retain the inner body biasing element 180 and the inner body 150 within the cavity of the housing. In such embodiments, the second end of the inner body biasing element 180 can engage against the cap 190.

The cap 190 may include a passage defining at least a portion of the second opening through the second end 112 of the housing. The passage through the cap 190 can have a cross-sectional width that is less than a cross-sectional width formed by the cavity of the housing such that the cap 190 can resist movement of the inner body biasing element 180 out of the cavity and permit the inner body to move out of the cavity through the passage of the cap 190.

In some aspects of the present disclosure the cap 190 can be unitarily formed with the housing 110 to form a ridge or flange of the housing that extends radially inward toward the longitudinal axis A1 of the housing.

To permit or resist a fluid flow through the luer protrusion, the coupling device 100 can include a valve 170. The valve 170 may be positioned in the cavity of the housing 110, and may be movable between the first end 114 and the second end 112 of the housing. The valve 170 can also be configured to move relative to the housing 110 and to the inner body 150, such that the valve 170 is movable when the inner body 150 is in any of the first and second positions.

The valve 170 may be movable between a closed position and an open position. In the closed position, the valve 170 can resist movement of a fluid through the coupling device 100. In some embodiments of the present disclosure, in the closed position, the valve 170 obstructs the valve passage 159 to resist movement of a fluid therethrough. In the open position, the valve 170 may be moved, relative to the inner body 150, to permit a fluid to flow through the inner body 150 and the valve 170. The closed and open positions of the valve 170 are shown, for example, in FIGS. 4 and 5, respectively.

The valve 170 may include a proximal end portion 171 and a distal end portion 172, where the distal end portion forms a post 174 and an arm 176. An inner surface of the valve 170 may define a fluid channel 178 extending through the proximal and distal end portions of the valve 170. In some aspects of the present disclosure, a portion of the fluid channel is formed by an opening 182 that extends radially between the inner and outer surfaces of the post 174.

The arm 176 of the valve may be configured to engage against a mating connector 20 such that the valve 170 moves in a direction from the closed position toward the open position when the mating connector 20 is coupled to the coupling device 100.

The arm 176 may extend along the post 174 and in a direction away from the proximal end portion 171 of the valve. The arm 176 can include a first segment 184 that extends radially outward in a direction away from the outer surface of the post, and a second segment 186 that extends from the first segment in a direction along the post and in direction away from the proximal end portion 171 of the valve.

In some embodiments of the present disclosure, the first segment 184 extends radially outward along a portion of the valve 170 between the proximal and distal end portions 171, 172. The first segment 184 may comprise a length that is greater than a thickness of the arm 176 such that the inner surface of the arm 176 is spaced apart from the outer surface of the post 174.

The valve 170 may be positioned in the cavity of the housing 110 with the post 174 extending into the valve passage 159 of the luer protrusion and the arm may be positioned so as to extend between the outer surface of the luer protrusion and the inner surface of the coupling sleeve. The proximal end portion 171 of the valve may extend through the passage 159 formed through the first end portion 154 of the inner body.

The outer surface of the post 174 may define a cross-sectional width that is less than a cross-sectional width defined by the inner surface of the luer protrusion 157 such that a gap is formed therebetween. The gap can permit a fluid between the outer surface of the post 174 and the inner surface of the luer protrusion 157, and through the opening 182.

In some embodiments, the post 174 comprises a protrusion 188 that extends radially outward to engage against the inner surface of the luer protrusion 157. The protrusion 188 may be configured to engage against the luer protrusion to resist movement of the post within the valve passage 159 in a direction that is non-linear or transverse relative to the longitudinal axis A2. In some aspects, the protrusion 188 can resist non-linear or transverse movement of the post 174 when the valve moved between the open and closed positions.

In the closed position of the valve 170, the post 174 may obstruct or intersect an opening through the luer protrusion 157 to resist a fluid flow therethrough. When the valve 170 is moved to the open position, the post 174 may be spaced apart from the opening of the luer protrusion 157, relative to when the valve 170 is in the closed position, to permit a fluid to move through the valve passage 159 of the luer protrusion and through the valve 170.

The arm 176, in some embodiments of the present disclosure, includes a cylindrical extension 192 that extends from the arm 176 in a direction away from the first end 114 of the housing. The cylindrical extension 192 can be configured to direct a force to arm 176 such that the valve 170 moves relative to the inner body 150 when the coupling device 100 is coupled to a mating connector. The cylindrical extension 192 can also be configured to engage against the luer protrusion of the inner body to form a seal between the cylindrical extension 192 and the luer protrusion 157. The seal formed between the cylindrical extension 192 and the luer protrusion 157 can resist unintended materials, such as fluids and/or solids, from moving into or out of the cavity of the housing 110.

The cylindrical extension 192 may extend from the second segment 186 of the arm to a terminal end 193 positioned proximal to the distal end of the luer protrusion 157. The cylindrical extension 192 and arm 176 can define a length between the first segment 184 and the terminal end 193 of the cylindrical extension.

In some embodiments of the present disclosure, the length of the cylindrical extension 192 and arm 176 are configured such that the terminal end 193 of the cylindrical extension is proximal to the distal end of the luer protrusion 157, and the distal most portion of the luer protrusion 157 extends beyond the terminal end 193 of the cylindrical extension, as shown in the detail view of FIG. 6. In such an embodiment, the terminal end 193 of the cylindrical extension is spaced apart from a plane P1 defined by the distal end of the luer protrusion 157.

In some embodiments of the present disclosure, the length of the cylindrical extension 192 and arm 176 are configured such that the terminal end 193 of the cylindrical extension is coplanar with or aligned with the plane P1 defined by the distal end of the luer protrusion 157, as shown in the detail view of FIG. 7.

In some embodiments of the present disclosure, the seal between the arm 176 and the luer protrusion 157 is formed by a valve seal 196. The valve seal 196 may be coupled to any of the arm 176 and the cylindrical extension 192, and may be configured to engage against the outer surface of the luer protrusion 157. The valve seal 196 may engage the outer surface of the luer protrusion 157 to maintain a seal as the valve 170 is moved between the closed and open positions.

The valve seal 196 may be coupled to the terminal end 193 of the cylindrical extension and extend radially inward in a direction toward the outer surface of the luer protrusion 157. The present disclosure contemplates that the valve seal 196 can be coupled to a portion of the arm 176 and/or the cylindrical extension 192 that is proximal to or spaced apart from the terminal end 193 of the cylindrical extension.

In some embodiments of the present disclosure, the valve seal 196 can include a proximal layer 197 and a distal layer 198, where each of the proximal and distal layers 197, 198 extend in a direction away from the cylindrical extension 192. Each of the proximal and distal layers 197, 198 comprise a length extending in a direction from the cylindrical extension 192 to an end of each respective layer. In some embodiments, such as is shown in FIG. 6, for example, the length L61 of the proximal layer 197 is approximately equal to the length L62 of the distal layer 198. In some embodiments, such as is shown in FIG. 7, for example, the length L61 of the proximal layer 197 is less that the length L63 of the distal layer 198.

In some embodiments of the present disclosure, an inner body seal 155 forms a seal between the inner body 150 and the valve 170 to resist unintended materials, such as fluids and/or solids, from moving into or out of the cavity of the housing 110.

To maintain a seal between the inner body 150 and the valve 170, the inner body seal 155 may be configured to move relative to any of the inner body 150 and valve 170. The inner body seal 155 may be coupled to an inner surface of the coupling sleeve 156 and may be configured to engage against any of the arm 176 and the cylindrical extension 192 to form the seal when valve 170 moves between the open and closed positions.

Although the inner body seal 155 can be coupled to the inner body 150, it should be understood that the present disclosure contemplates that the inner body seal 155 can be coupled to the valve 170 in some embodiments.

The proximal end portion 171 of the valve can be fluidly coupled to the tubing 16 to permit a fluid flow through the fluid channel 178 and tubing 16. The tubing 16 extend through the first end 114 of the housing and be coupled to the proximal end portion 171 of the valve. In some embodiments, another length of tubing or a bellows is positioned between the proximal end portion 171 of the valve and the first end 114 of the housing, and the tubing 16 can be coupled to the first end 114 of the housing.

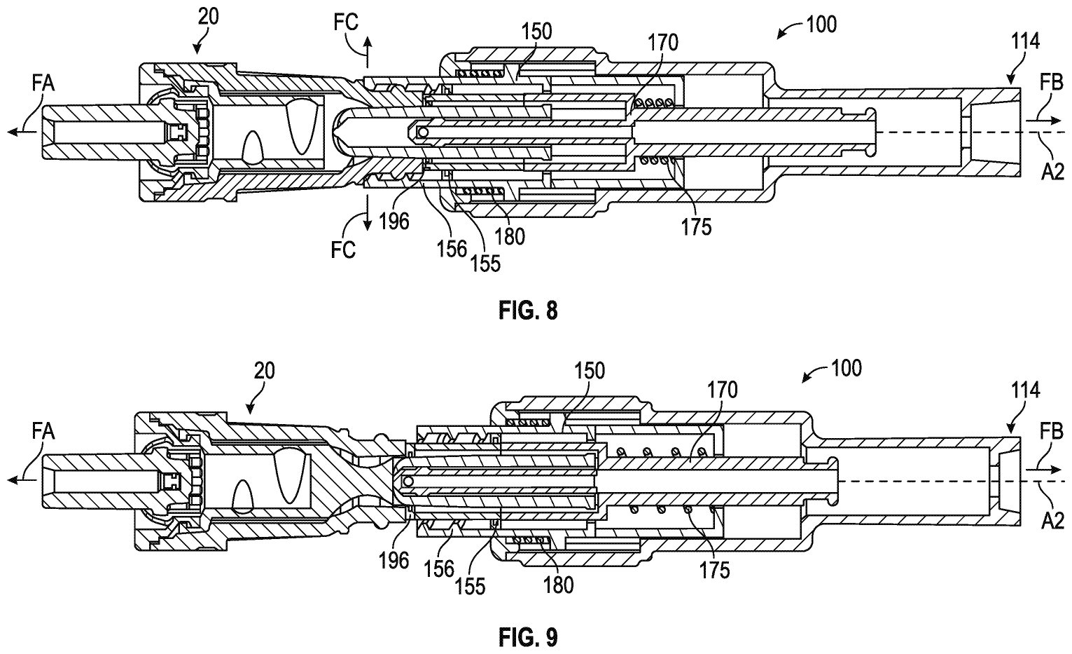

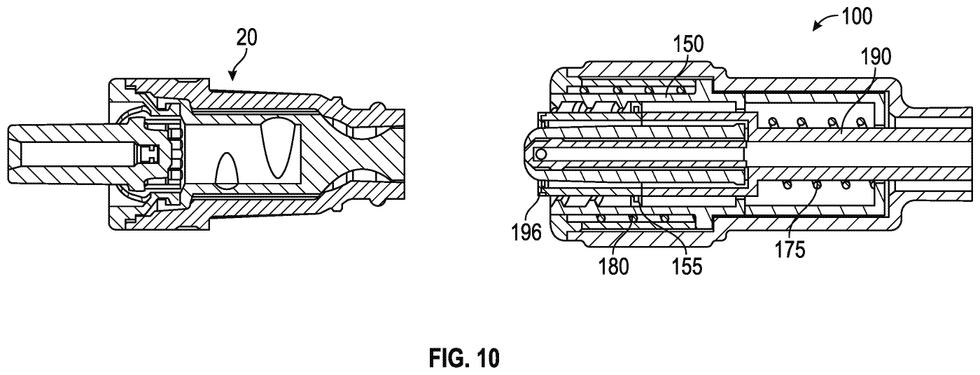

The coupling device 100 and a mating connector 20 are shown together in FIGS. 8-10, where the coupling device 100 is in the second configuration in FIGS. 8 and 9, and in the first configuration in FIG. 10.

The coupling device 100 may be moved to the second configuration after a pulling or tension force FA, FB exceeds a first threshold of the coupling device 100. When the mating connector 20 is coupled with the coupling device 100, and a pulling or tension force exceeds a threshold of the coupling device 100, the mating connector 20 moves with the inner body 150 relative to the housing 110. As the coupling device 100 is moved to the second configuration, the inner body 150 is moved in a direction away from the first end 114 of the housing, and the inner body biasing element 180 is compressed between the inner body 150 and the housing 110, as shown in FIG. 8.

In the second configuration, the mating connector 20 can remain coupled to the inner body 150 such that the valve 170 remains in the open position and a fluid can flow between the mating connector 20 and the coupling device 100. In some embodiments of the present disclosure, a valve biasing element 175 is compressed between the valve 170 and a portion of the inner body 150 when the valve is in the open position.

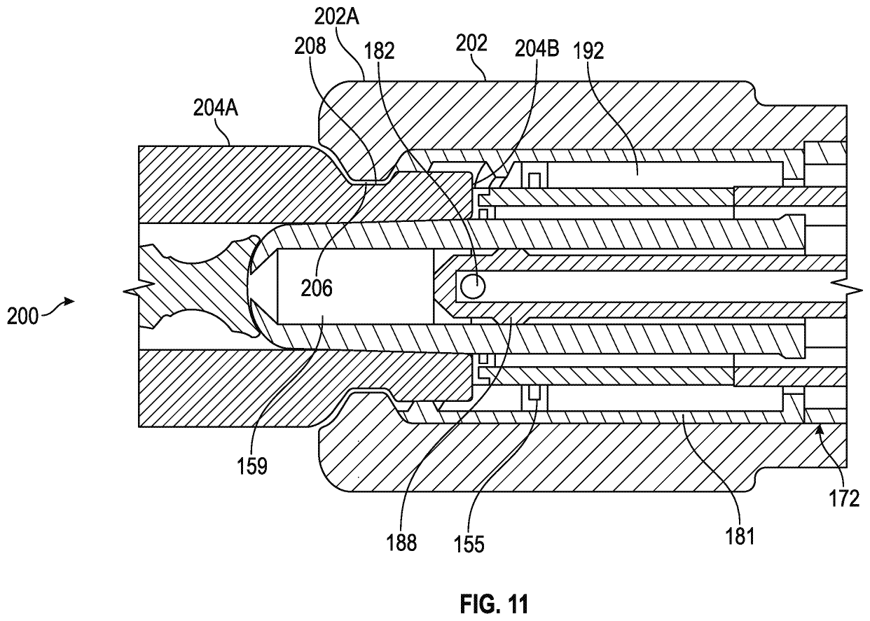

In some embodiments, as illustrated in FIG. 11, the connecting mechanism 200 may include a first connecting member 202 and a second connecting member 204. The first connecting member 202 may include a first end 202A and a second end 202B opposite the first end 202A. The first connecting member 202 may be coupled to the connector housing 110 proximate the first end 202A of the first connecting member 202. The second end 202B of the first connecting member 202 may include a projection 206. The projection 206 may extend from a surface of the connecting member 202. In some embodiments, the projection 206 may be disposed at an angle of approximately 45 degrees relative the surface of the first connecting member 202.

The second connecting member 204 may include a first end 204A and a second end 204B. The second connecting member 204 may be coupled to the mating connector 20 proximate the first end 204A. The second end 204B of the second connecting member 204 may include a groove 208. The groove 208 may be sized and shaped to receive the projection 206.

When the connectors 10, 20 are coupled, the first connecting member 202 and the second connecting member 204 may be coupled such that the projection 206 is received within the groove 208. When coupled, the connection between the projection 206 and the groove 208 may be strong enough to maintain the coupling of connectors 10, 20 until pulling or tension force FA, FB that exceeds the threshold is applied to one or both of the connectors 10, 20. In some embodiments, the predetermined threshold force is approximately 5 pounds (lbs). The predetermined threshold force may be from approximately 1 lb to approximately 8 lbs, approximately 3 lbs to approximately 7 lbs, approximately 4 lbs to approximately 6 lbs, or greater than 8 lbs. When a pulling or tension force FA, FB that exceeds the threshold is applied to one or both connectors 10, 20, the projection 206 may be decoupled from the groove 208 such that the first connecting member 202 and the second connecting member 204 are decoupled. As a result, the connectors 10, 20 may be decoupled.

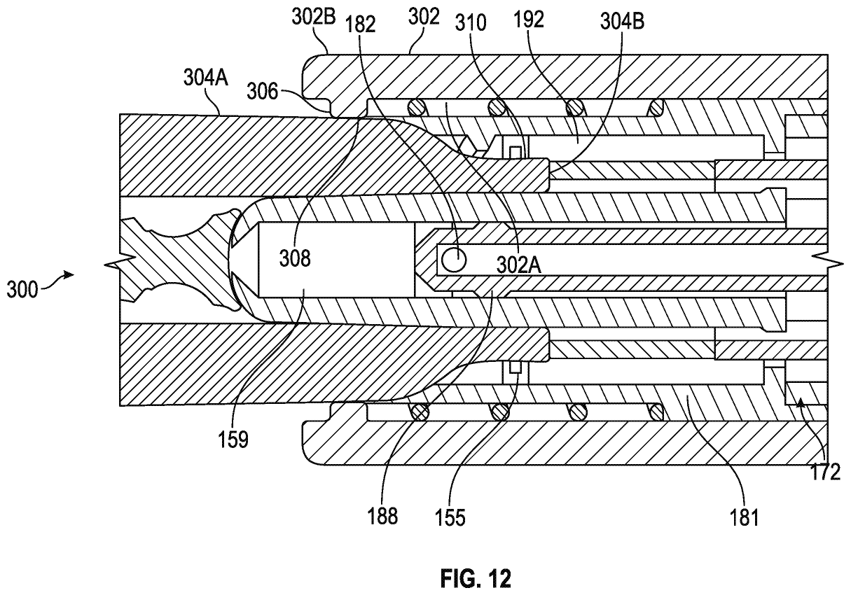

In some embodiments, as illustrated in FIG. 12, the connecting mechanism 300 may comprise a first connecting member 302 and a second connecting member 304. The first connecting member 302 may be formed as press ring and may include a first end 302A and a second end 304B opposite the first end 302A. The first connecting member 302A may be coupled to the connector 20 proximate the first end 302A and may further comprise a projection 306 disposed proximate the second end 302B. The projection 306 may extend from a surface of the first connecting member 302. In some embodiments, the projection 304 may include an substantially flat engaging surface 308. However, the flat engaging surface 308 need not be so limited and may include an engaging surface having a varied profile. The first connecting member 302 may be configured to be flexible such that it may flex and/or stretch to a certain degree.

The second connecting member 304 may include a first end 304A and a second end 304B opposite the first end 304A. The second connecting member 304 may be coupled to the connector 10 at the first end 304A. The second end 304B of the second connecting member 304 may include an engaging surface 310. The engaging surface 310 may be substantially flat.

When the connectors 10, 20 are coupled, the first connecting member 302 and the second connecting member 304 may be coupled. In particular, the projection 306 may engage a surface of the second connecting member 304 such that the engaging surface 308 contacts a surface of the second connecting member 304. The first connecting member 302 may be biased toward the second connecting member 304 such that, when coupled, the engaging surface 308 applies pressure to the surface of the second connecting member 304. As such, the first connecting member 302 and the second connecting member 304 may aid in maintaining the connection between the connectors 10, 20 until a pulling or tension force FA, FB that exceeds a threshold is applied to one or both of connectors 10, 20. In the event of such a pulling or tension force FA, FB, a surface of the first connecting member 302 and a surface of the second connecting member 304 translate or slide along one another in opposite directions until the first connecting member 302 and the second connecting member 304 are decoupled.

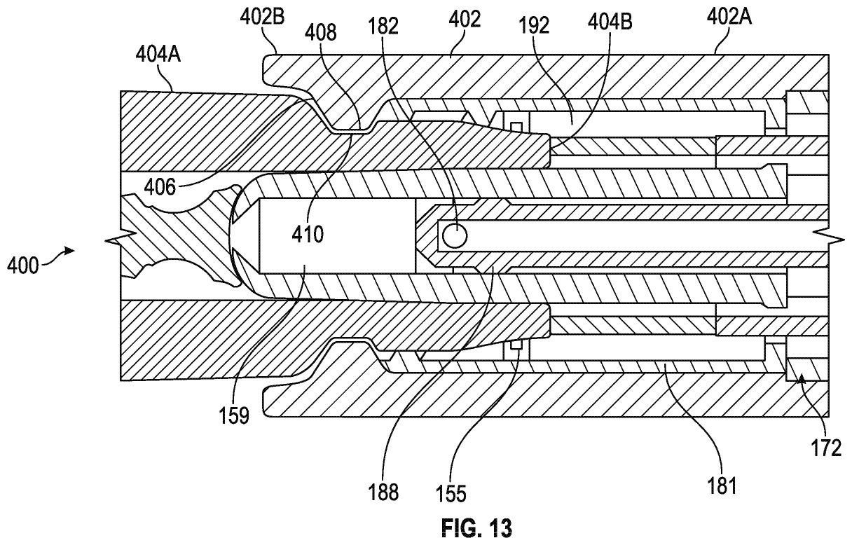

In some embodiments, as illustrated in FIGS. 13, the connecting mechanism 400 may include a first connecting member 402 and a second connecting member 404. The connecting mechanism 400 may be similar to the connecting mechanism 300 described above. In particular, the connecting mechanism 400 may be configured as a press ring and may further comprise a groove or detent 410. Similar to the connecting mechanism 300, the first connecting member 402 may be coupled to the connector 10 at a first end 402A and comprise a projection 406 disposed proximate a second end 402B opposite the first end. The projection 404 may extend from a surface of the first connecting member 402. In some embodiments, the projection 404 may include a substantially flat engaging surface 408. However, the flat engaging surface 408 need not be so limited and may include an engaging surface having a varied profile. The first connecting member 402 may be configured to be flexible such that it may flex and/or stretch to a certain degree. Moreover, the second connecting member 404 may be coupled to the connector 20 at a first end 404A and may include a groove or detent 410 proximate a second end 404B. The groove or detent 410 may be sized and shaped to receive the projection 406 of the first connecting member 402.

Similar to connecting mechanisms 200, 300 described above, the first connecting member 402 and the second connecting member 404 may aid in maintaining the connection of connectors 10, 20 until a pulling or tension force FA, FB that exceeds threshold is applied to one or both connectors 10, 20. When a pulling or tension force FA, FB that exceeds the threshold is applied to one or both connectors 10,20, the projection 406 may be removed from the groove or detent 410 such thereby allowing the first connecting member 402 and the second connecting member 404 to translate or slide with respect to one another in opposite directions until the first connecting member 402 and second connecting member 404 are decoupled, thus allowing the connectors 10, 20 to decouple.

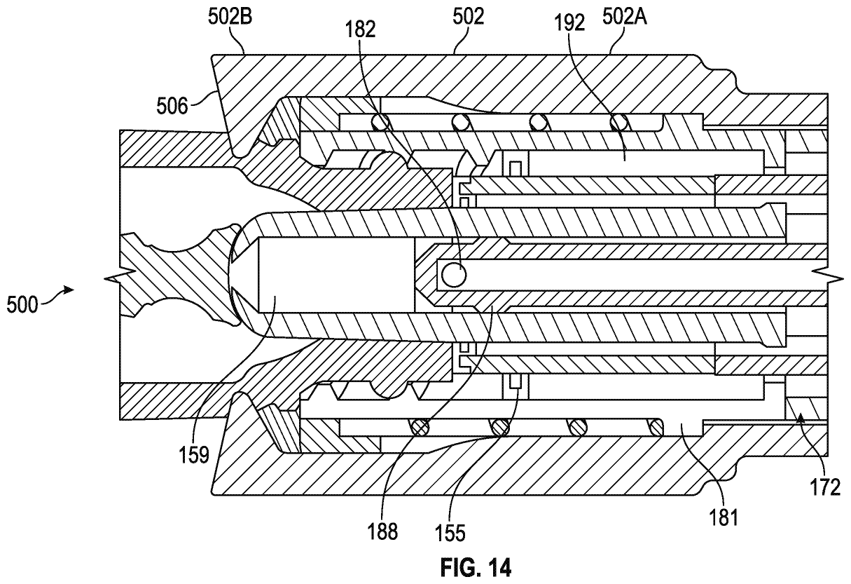

In some embodiments, as illustrated in FIG. 14, the connecting mechanism 500 may include a first connecting member 502 coupled to one of connectors 10, 20. The first connecting member 502 may be coupled at a first end 502A to one of connectors 10, 20. The first connecting member 502 may include a projection 504 disposed proximate a second end 502B opposite the first end 502A. The projection 504 may be formed to have a tooth-like configuration. In particular, the projection 504 may extend outwardly from a surface of the first connecting member 502 and may include a sloped portion 506 extending toward the second end 502B of the first connecting member 502 thereby creating a stepped surface. The first connecting member 502 may be configured to engage an elastomer ring (not shown) disposed on the other of the connectors 10,20.

The coupling of the first connecting member 502 and the elastomer ring (not shown) may aid in maintaining the coupling of connectors 10, 20 until a pulling or tension force FA, FB exceeds threshold is applied to one or both connectors 10, 20. When such pulling or tension force FA, FB is applied to one or both connectors 10, 20, the projection 504 may slip against the elastomer ring thereby enabling disconnection of the connectors 10, 20.

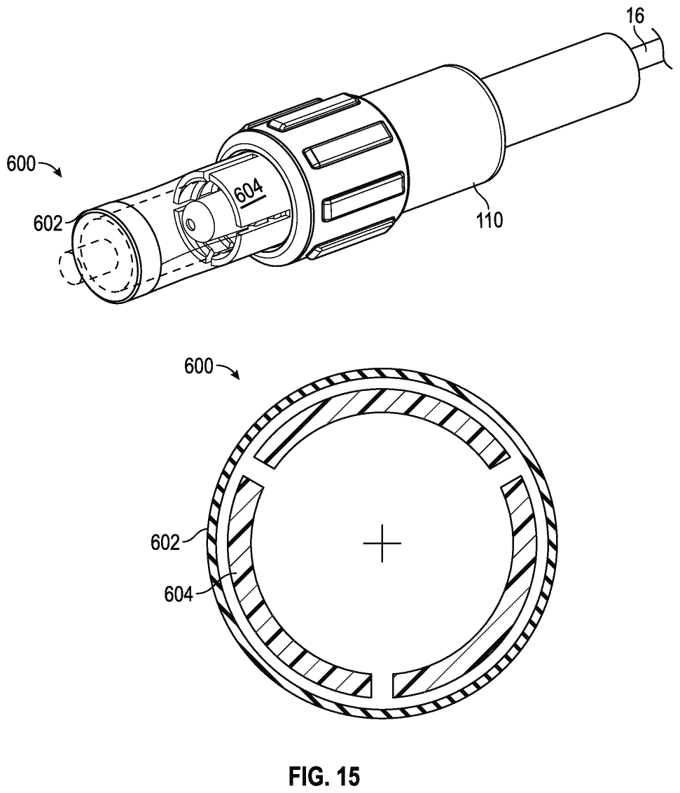

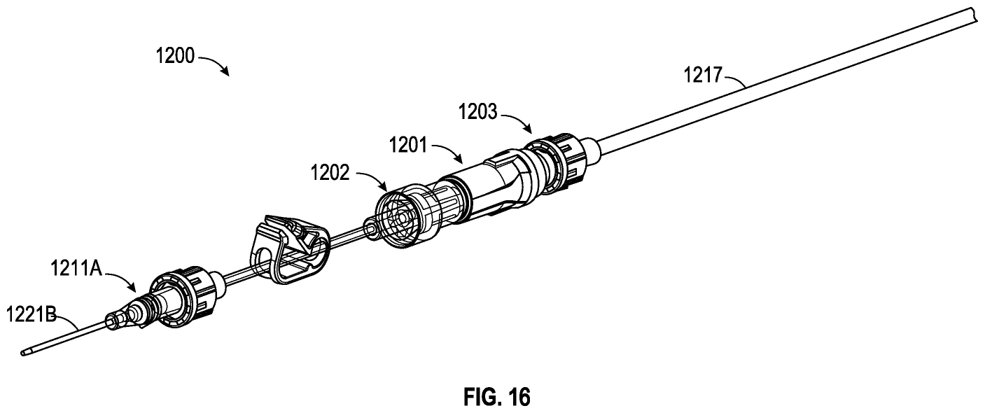

In some embodiments, as illustrated in FIGS. 15, the connecting mechanism 600 may comprise a first connecting member 602 and a second connecting member 604. The connecting mechanism 602 may be coupled to connector 10 or connector 20. The first connecting member 602 may be an elastic band and the second connecting member 604 may be one or more threads. The threads 604 may be split to allow for deflection and the elastic band 602 may be disposed around the threads 604 thereby providing radial support to the threads 604. Moreover, the elastic band 602 may be molded so as to have different durometers and/or thicknesses thereby allowing for varying disconnection force levels.

Similar to the connecting mechanisms, 200, 300, 400, 500, when the elastic band 602 is positioned around the threads 604, the connecting mechanism 600 may aid in maintaining the connection of the connectors 10, 20. In the event that a pulling or tension force FA, FB exceeds a threshold is applied to one or both connectors 10, 20, the elastic band 602 may expand to allow the threads 604 to pull thereby allowing for the disconnection of connectors 10, 20.

When the mating connector 20 separates from the coupling device 100, as described above, the valve 170 can move from the open position (FIG. 8) to the closed position such that the valve passage 159 is obstructed and movement of a fluid therethrough is resisted, as shown in FIG. 9. In some embodiments, the valve biasing element 175 expands to exert a force on the valve 170 to move the valve from the open position toward the closed position.

After the pulling or tension force FA, FB is reduced to less than the first threshold, the inner body 150 can move from the second position to the first position so that the coupling device 100 is in the first configuration as shown in FIG. 10. The inner body 150 can move to the first position so that the coupling device 100 is in the first configuration when the pulling the tension force FA, FB is reduced or when the mating connector 20 is separated from the coupling device 100 such that the pulling the tension force FA, FB is no longer present.

In some embodiments, the coupling device may be removed and replaced after occurrence of a disconnection event described above. In other words, when the connecting mechanism 200, 300, 400, 500, 600 is decoupled and, thus, the coupling device is disconnected from the mating connector, the coupling device, or portions thereof, may be removed and replaced thereby allowing for a user to replace the coupling device with a sanitized coupling device.

The coupling device 1200 may include a body portion 1205, a proximal connector 1214 disposed at least partially in the body portion 1205, and a distal connector 1212 slidably coupled to the body portion 1205. The body portion 1205 may have an inner surface defining a cavity 1208 terminating in an open end 1262 of the body portion 1205, and a luer portion 1227 extending in the cavity 1208 and through the open end 1262 of the body portion 1205. The inner surface of the body portion 1205 may include a plurality of notches 1282 recessed therein. In some embodiments, the body portion 1205 may include a plurality of wings 1210. In some embodiments, the plurality of wings 1210 may extend from an outer surface of the proximal connector 1214 to an outer surface 1215 of the distal connector 1212. As shall be described in further detail below with respect to the operation of the connector assembly 1201, the distal connector 1212 may be slidably coupled to the proximal connector 1214 via the plurality of wings 1210. In some embodiments, each of the plurality of wings 1210 may have a proximal end 1260 and a distal end forming the open end 1262. Each of the wings 1210 may have an inner surface 1264 including a first linear portion 1266 extending proximally from the open end 1262, a ramp portion 1252 extending proximally and radially outward from the first linear portion 1266, and a second linear portion 1270 extending proximally from the ramp portion 1252.

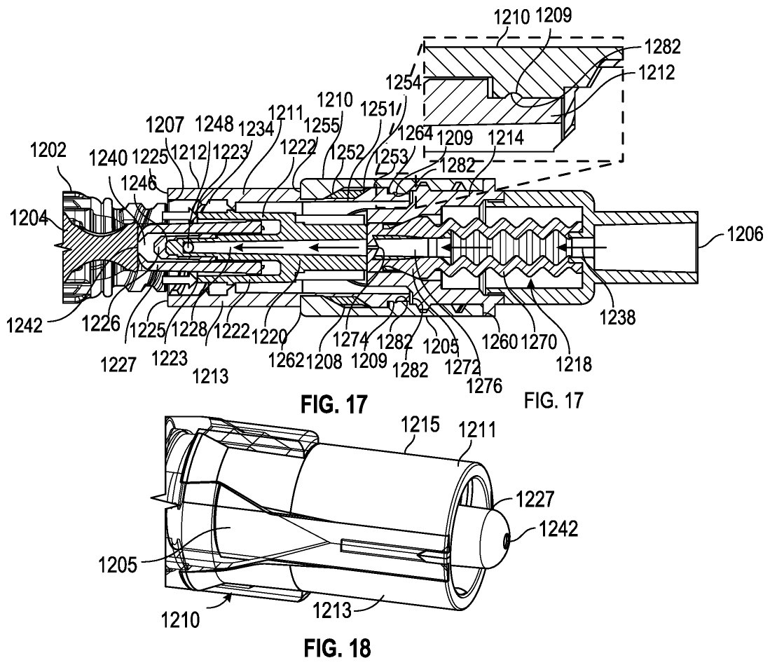

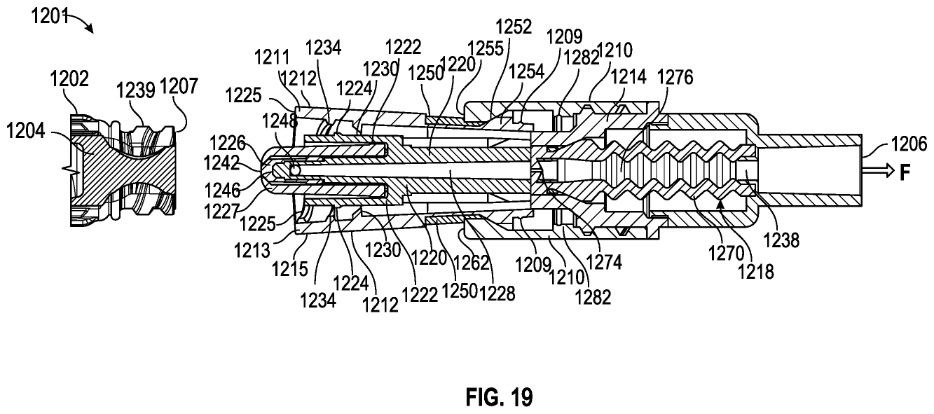

The distal connector 1212 may have first and second arms 1211 and 1213 that are pivotable relative to each other (illustrated in FIG. 17). For example, in some embodiments, the first and second arms 1211 and 1213 may each include a pair of hinge portions having coupling apertures. The hinge portions and respective coupling apertures of the first and second arms 1211 and 1213 may be aligned coaxially such that pivot pins may be inserted into the aligned coupling apertures at opposing sides of the first and second arms 1211 and 1213. Accordingly, the pair of hinge portions of the first arm 1211 may be rotatably coupled to the pair of hinge portions of the second arm 1213, thereby pivotably coupling the first and second arms 1211 and 1213 of the distal connector 1212 to each other.

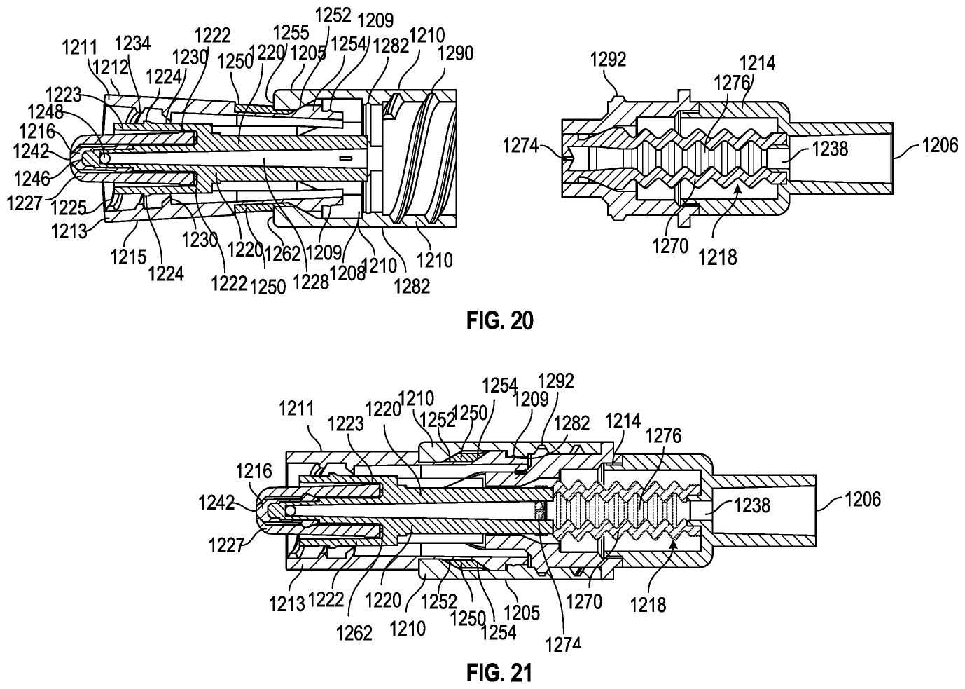

The distal connector 1212 may have first and second arms 1211 and 1213 that are pivotable relative to each other (illustrated in FIG. 20). For example, in some embodiments, the first and second arms 1211 and 1213 may each include a pair of hinge portions having coupling apertures. The hinge portions and respective coupling apertures of the first and second arms 1211 and 1213 may be aligned coaxially such that pivot pins may be inserted into the aligned coupling apertures at opposing sides of the first and second arms 1211 and 1213. Accordingly, the pair of hinge portions of the first arm 1211 may be rotatably coupled to the pair of hinge portions of the second arm 1213, thereby pivotably coupling the first and second arms 1211 and 1213 of the distal connector 1212 to each other.

In some embodiments of the present disclosure, the ability to pivot the first and second arms 1211 and 1213 is provided by the body portion 1205 having a pivot bore or a pivot pin, and the first and second arms 1211 and 1213 having the other of the pivot bore or the pivot pin. The pivot bore can be located on opposite sides of the body 1205, and each of the first and second arms 1211 and 1213 can have a pair of pivot pins configured to be received into the pivot bore.

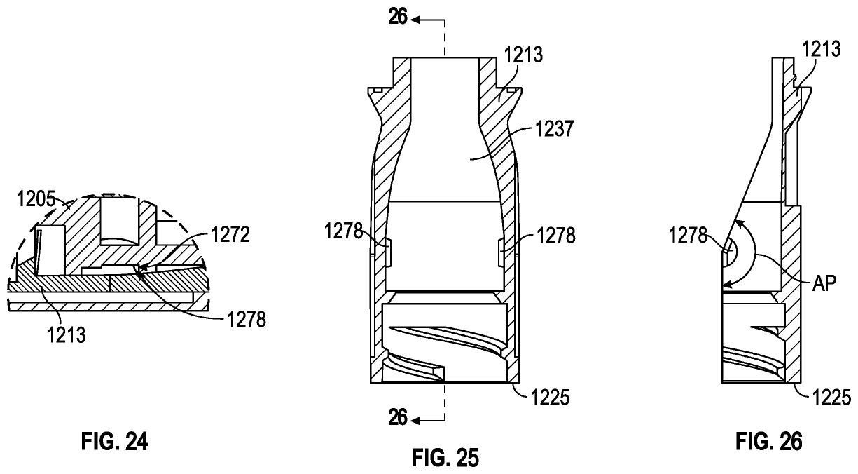

Referring to FIGS. 24 and 25, which show a cross-sectional view of the connector assembly 1201 in the direction A-A and a detail view thereof, the body portion 1205 includes a pivot bore 1272 and each of the first and second arms 1211 and 1213 include a pivot pin 1278 configured to be positioned within the pivot bore 1272.

The second arm 1213 is shown in FIG. 26 and shows an inner surface 1237 of the distal connector 1212 which defines a cavity 1243 terminating in an open end 1225 of the distal connector. The second arm 1213 includes first and second pivot pins 1278 each located along lateral side portions of the second arm such that the second arm 1213 is pivotable about an axis that extends between the first and second pivot pins 1278. In some aspects of the present disclosure, the first and second pivot pins 1278 are located diametrically opposite to each other. Although the second arm 1213 is shown in isolation for clarity, it should be understood that the present disclosure contemplates that the first arm 1211 can have the same or similar features as the second arm 1213.

With a pivot pin 1278 of each of the first and second arms 1211 and 1213 positioned in a pivot bore 1272, a partial circular cross-section of each pivot pin 1278 having a perimeter that is less than 180 can permit each of the first and second arms 1211 and 1213 to pivot relative to the body potion 1205. In some embodiments of the present disclosure, each pivot pin has a circular perimeter that extends about an angle AP around an axial center of the pivot pin 1278. In some embodiments, the angle AP is between approximately 10 degrees and approximately 180 degrees. In some embodiments, the angle AP is between approximately 90 degrees and approximately 175 degrees. In some embodiments, the angle AP is between approximately 120 degrees and approximately 160 degrees. In the embodiment shown in FIG. 28, the angle AP is 158 degrees.

In some embodiments of the present disclosure, proximal and distal ends of the first and second arms 1211 and 1213 are configured to move toward and away from the body portion 1205 when any of the first or second arms 1211 are 1213 are pivoted. To permit the proximal and distal ends of the first and second arms 1211 and 1213 to move toward and away from the body portion 1205, the pivot pins are located between the proximal and distal ends of the first and second arms 1211 and 1213. Movement of the proximal and distal ends of the first and second arms 1211 and 1213 toward and away from the body portion 205 can also be permitted by the lateral sides of each of the first and second arms 1211 and 1213 having a chamfer at the proximal end portion thereof. In another aspect, when the first and second arms 1211 and 1213 are coupled to the pivot bore, a space is created between the first and second arms 1211 and 1213, from the pivot pin toward the proximal ends of the first and second arms 1211 and 13.

When the distal connector 1212 moves in a distal direction, relative to the proximal connector 1214, the first and second arms 1211 and 1213 pivot so that the distal ends of the first and second arms 1211 and 1213 are moved radially outward. When the distal ends of the first and second arms 1211 and 1213 are moved radially outward, the mating connector 1202 can be decoupled from the distal connector.

As illustrated, the first and second arms 1211 and 1213 may define (i) the outer surface 1215 of the distal connector 1212, and (ii) an inner surface 1237 of the distal connector 1212 which defines a cavity 1243 terminating in an open end 1225 of the distal connector 1212. The inner surface 1237 of the distal connector 1212 may have at least one stop 1230 (illustrated in FIG. 4) extending radially inward from the inner surface 1221. As depicted, when the mating connector 1202 is coupled to the distal connector 1212, each protrusion 1224 may abut a corresponding one of the at least one stops 1230 to prevent unintended expansion of the compressible valve member 1218 and distal movement of the post 1220.

As further depicted, each of the first and second arms 1211 and 1213 may include at least one detent 1209 extending radially outward from the outer surface 1215 of the distal connector 1212. When the distal connector 1212 is coupled to the mating connector 1202, the detents 1209 of the first and second arms 1211 and 1213 engage the plurality of notches 1282 to prevent decoupling of the mating connector 1202 from the distal connector 1212 when a force F applied to the proximal connector 1214 (i.e., a proximal-direction force) is less than or equal to a predetermined proximal threshold (pullout) force.

In some embodiments, the outer surface 1215 defined by the first and second arms 1211 and 1213 of the distal connector 1212 may have a slot 1250 recessed at least partially therein. The slot 1250 may have a linear surface 1251 extending proximally from a distal end 1255 of the slot 1250, and a ramp surface 1254 extending from the linear surface 1251 to a proximal end 1253 of the slot 1250. As depicted, the ramp portion 1252 of each wing 1210 may be parallel to the ramp surface 1254 of each slot 1250 and the first linear portion 1266 of each wing 1210 may be parallel to the linear surface 1251 of each slot 1250. When the force F applied to the proximal connector 1214 exceeds the predetermined threshold, the detents 1209 may disengage from the notches 1282, causing the distal connector 1212 to translate distally relative to the proximal connector 1214. Upon disengagement of the detents 1209 from the notches 1282, the ramp surfaces 1254 of the slots 1250 may push against the ramp portions 1252 of the wings 1210 to pivot the first and second arms 1211 and 1213 radially outward and widen the open end 1225 of the distal connector 1212 to release the mating connector 1202 (as illustrated in FIG. 20).

In operation, the connector assembly 1201 may generally be coupled to the mating or reciprocal connector 1202 at a distal end thereof and be coupled to the mating luer connector 1203 at a proximal end thereof. In some embodiments, the mating or reciprocal connector 1202 may be a needleless connector, and the mating luer connector 1203 may be a male luer connector. For example, as depicted in FIG. 18, mating connector 1202 may be inserted and coupled into the open end 1225 of the distal connector 1212, and the mating luer connector 1203 may be inserted and coupled to the inlet port 1206 of the proximal connector 1214. In some embodiments, the mating connector 1202 may include threads 1239 for mating with complimentary threads 1175 in the open end 1125 of the distal connector 1212. When the distal connector 1212 is coupled to the mating connector 1202, the at least one detent 1209 engages the at least one notch 1282 to prevent decoupling of the mating connector 1202 from the distal connector 1212 at or below a predetermined proximal threshold (pullout) force applied to the proximal connector 1214.

FIG. 18 depicts the coupled configuration of the mating connector 1202 and the distal connector 1212 of the connector assembly 1201 when the force F below or equal to the predetermined proximal threshold (pullout) force is applied to the proximal connector 1214. For example, as described above with respect to the coupling device 100, the connector assembly 1201 may be subject to proximal-direction pullout forces as a result of patients rolling over in bed, patients catching tubing or lines on bed rails, moving patients to a different bed, fidgeting by pediatric patients, and/or disoriented adult patients tugging on their lines. When subject to lower pullout forces, e.g., forces F below or equal to the predetermined proximal threshold (pullout) force, the connector assembly 1201 may be configured so as to retain the mating connector 1202 within the distal connector 1212 such that the fluid path 1238 remains open and medical fluid, e.g., IV fluid may be administered from the fluid line or tubing 1217. In particular, as illustrated in FIG. 18, when the distal connector 1212 is coupled to the mating connector 1202, the closed end 1246 of the post 1220 (along with the attached seal 1226) may be displaced away from the outlet port 1242 of the luer portion 1227 to permit flow through the fluid path 1238 via the luer portion 1227.

As depicted, in the coupled configuration of the mating connector 112202 and the distal connector 1212, the luer portion 1227 may extend through a top surface 1207 of the mating connector 1202 into a mating luer of the mating connector 1202 to displace a flexible valve 1204 of the mating connector 1202. Accordingly, when the mating connector 1202 is coupled to the distal connector 1212, the mating connector 1202 may exert a force to urge the plurality of arms 1222 of the post 1220 proximally, which in turn may cause the post 1220 to compress the compressible valve member 1218 proximally and displace the closed end 1246 of the post 1220 away from the outlet port 1242 of the luer portion 1227. Due to the compression, the slit 1274 at the distal end portion of the compressible valve member 1218 may be opened, thereby fluidly communicating the internal chamber 1276 of the compressible valve member 1218 with the lumen 1228 of the post 1220 and permitting flow through the fluid path 1238 into the mating connector 1202 via the outlet port 1242 of the luer portion 1227. For example, as illustrated in FIG. 18, the plurality of arms 1222 and post 1220 may be forced away from the outlet port 1242 of the luer portion 1227 by the top surface 1207. In the open position, the closed end 1246 of post 1220 is displaced from the outlet port 1242 of the luer portion 1227, thereby permitting the medical fluid to flow between the outlet port 1242 of the luer portion 1227 and the lumen 1228 of the post 1220 through the passage 1248.

FIG. 20 illustrates an operational view of the connector assembly 1201 decoupled from the mating connector 1202 due to pivoting open of the distal connector 1212 of the connector assembly 1201 when the connector assembly 1201 is subject to the proximal-direction force F above the predetermined threshold, in accordance with some embodiments of the present disclosure. According to various embodiments of the present disclosure, when subject to higher pullout forces, e.g., forces F exceeding the predetermined proximal threshold (pullout) force, the connector assembly 1201 may be configured so as to release or otherwise decouple the mating connector 1202 from within the distal connector 1212 at which point the fluid path 1238 may close and the medical fluid, e.g., IV fluid may be discontinued from entering the fluid line or tubing 217. As described above, the predetermined proximal threshold (pullout) force) may be approximately 5 lbs.).

In particular, as illustrated in FIG. 20, when the force applied to the proximal connector 1214 exceeds the predetermined threshold, the detents 1209 may disengage from the notches 1282 to allow the distal connector 1212 to translate distally relative to the proximal connector 1214 and pivot the first and second arms 1211 and 1213 radially outward to decouple the mating connector 1202 from the distal connector 1212, as shall be described in further detail below. For example, in operation when the proximal pullout force F applied to the proximal connector 1214 exceeds the predetermined threshold, the pullout force F may cause the detents 1209 of the first and second arms 1211 and 1213 to disengage or otherwise be displaced from the notches 1282 of the wings 1210. Upon disengagement of the detents 1209 from the notches 1282, the compressed valve member 1218 may expand and correspondingly move the post 1220 distally. As the post 1220 moves distally, the protrusion 1224 on each of the plurality of arms 1222 of the post 1220 may exert a distal force on the corresponding stop 1230 which it abuts, thereby causing the distal connector 1212 to move or translate distally. In particular, as illustrated in FIG. 20, as the compressible valve member 1218 expands, the first linear portion 1266 of each of the wings 1210 may slide along the linear surface 1251 of the slot 1250 to allow the distal connector 1212 to translate distally relative to the proximal connector 1214. The distal connector 1212 may continue to translate distally as each of the wings 1210 continue to slide along the linear surface 1251 of each slot 1250 up to a point where the ramp surface 1254 of each slot 1250 contacts and abuts the ramp portion 1252 of each wing 1210. As the compressible valve member 1218 continues to expand, the ramp surface 1254 of each slots 1250 may push against the ramp portions 1252 of the wings 1210 to pivot the first and second arms 1211 and 1213 radially outward and widen the opening at the open end 1225 of the distal connector 1212. As the compressible valve member 1218 continues to expand distally, the compressible valve member 1218 may continue to exert a force on the post 1220 which moves or otherwise displaces the closed end 1246 of the post 1220 towards the outlet port 1242 of the luer portion 1227 to seal the outlet port 1242.

As depicted, as the first and second arms 1211 and 1213 may continue to pivot radially outward, the mating connector 1202 may then be released and completely decoupled from the distal connector 1212. In this state, the outlet port 1242 of the luer portion 1227 is sealed by the seal 1226 at the closed end 1246 of the post 1220, thereby closing the fluid path 1238 and advantageously preventing microbial ingress into the fluid path 1238. Further advantageously, the sealed outlet port 1242 may prevent the medical fluid, e.g., IV fluid from further exiting or otherwise spilling out of the fluid path 238 via the outlet port 1242 of the luer portion 1227 upon the disconnection of the mating connector 1202.

Accordingly, the first and second arms 1211 and 213 are advantageously be designed to release the mating needleless connector 1202 when a pullout force F exceeding the threshold pullout force is applied to the tubing 1217 and the proximal connector 214. As such, both the connector assembly 1201 (for example, but not limited to a Texium valve) and the mating connector 1202 (for example, but not limited to a Smartsite valve) may automatically shut off at separation thereby preventing (i) microbial ingress into the fluid path 138, and (2) leakage or spillage of medical fluids upon accidental disconnection by higher pullout forces exceeding the predetermined threshold force. The aforementioned configuration is advantageous over currently existing catheter dislodgement devices or couplers which may or may not be generally adhesive based, and capable of only preventing catheter dislodgement at lower pullout forces. These currently existing catheter dislodgement devices or couplers are not capable of preventing catheter dislodgement at higher pullout forces, but instead may release in response to higher pullout forces (for example forces exceeding 5 lbs.) experienced during patients rolling over in bed, patients catching tubing or lines on bed rails, moving patients to a different bed, fidgeting by pediatric patients, and/or disoriented adult patients pulling out their lines.

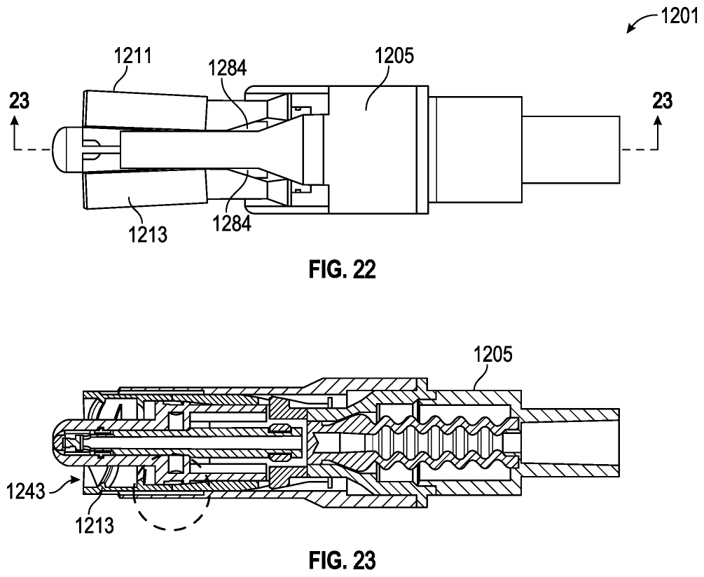

FIG. 21 illustrates a cross-sectional operational view of the proximal connector 1214 of the connector assembly 1201 decoupled from the connector assembly 1201 for swabbing, in accordance with some embodiments of the present disclosure. FIG. 22 illustrates a cross-sectional operational view of the proximal connector 1214 of the connector assembly coupled to the rest of the connector assembly 1201 after swabbing, in accordance with some embodiments of the present disclosure. According to various embodiments of the present disclosure, the first and second arms 1211 and 1213 of the distal connector 1212 may be returned from the pivoted state illustrated in FIG. 21 to the non-pivoted state illustrated in FIG. 22 by pinching together or otherwise exerting opposite-direction forces on the first and second arms 1211 and 1213 to pivot the first and second arms 211 and 213 radially inward to the original state prior to the radially-outward pivoting.

As illustrated in FIG. 21, the proximal connector 1214 may be decoupled or otherwise detached from the rest of the connector assembly 1201. For example, in the coupled or assembled configuration of the connector assembly 1201, the proximal connector may be coupled in the cavity 1208 of the body portion 1205. As depicted, the inner surface of the body portion 1205 may include a plurality of threads 1290, and the outer surface of the proximal connector 214 may include complimentary threads 1292 for engaging with the threads 1290 of the body portion. In order to decouple or otherwise detach the proximal connector 1214 from the rest of the connector assembly 1201, the proximal connector 1214 may be unthreaded or otherwise unscrewed and removed from the cavity 1208 of the body portion 1205. Although the decoupling of the proximal connector 1214 from the rest of the connector assembly 1201 is described herein with respect to a threaded engagement, the various embodiments are not limited to the aforementioned configuration, and the proximal connector 1214 may instead be removably coupled to the body portion by any other suitable coupling or fastening method.

The aforementioned configuration in which the proximal connector 1214 is removably coupled to the rest of the connector assembly is advantageous in that the luer portion of the proximal connector 1214, one removed from the cavity 1208 of the body portion 1205, may then be swabbed or otherwise disinfected. In some embodiments, the rest of the connector assembly 1201 (i.e., the body portion 1205 and distal connector 1212 assembly) may be discarded and replaced with a new and sterile body portion 1205 and distal connector 1212 assembly without breaching or otherwise infecting the fluid path 1238. As illustrated in FIG. 22, the proximal connector 1214 may then be attached or coupled to the new and sterile body portion 1205 and distal connector 1212 assembly.

Similarly, when decoupled or otherwise detached from the connector assembly 1201, the mating connector 1202 may also be swabbed or otherwise disinfected. The mating connector 1202 may then be reconnected to the connector assembly 1201 as illustrated in FIG. 18. For example, after disinfection of the mating connector 1202, the mating connector 1202 may then be coupled or connected to the new sterile connector assembly 1201. The mating connector 1202 may be advanced towards the luer portion 1227 of the distal connector 1212. As the mating connector 1202 is advanced towards the luer portion 1227 of the distal connector 1212, the luer portion 1227 may advance into the interior of the mating connector 1202 and compress the valve member 1204 of the mating connector 1202. As the luer portion 1227 advances further into the interior of the mating connector 1202, the top surface 1207 of the mating connector 1202 may exert a force to urge the plurality of arms 1222 of the post 1220 proximally, which in turn may compress the compressible valve member 1218 and displace the closed end 1246 of the post 1220 away from the outlet port 1242 of the luer portion 1227, as illustrated in FIG. 18. For example, as previously discussed the plurality of arms 1222 and post 1220 may be forced away from the outlet port 1242 of the luer portion 1227 by engagement of a distal end portion 1223 of the arms 1222 against the top surface 1207 of the mating connector 1202. In the open position, the closed end 1246 of post 1220 is displaced from the outlet port 1242 of the luer portion 1227, thereby re-opening the fluid path 1238, and permitting the medical fluid to flow through the outlet port 1242 of the luer portion 1227 and into the mating connector 1202 via the lumen 1228 and the passage 1248 of the post 1220. Accordingly, administration of the IV fluid from the fluid line or tubing 1217 to the catheter 1221B via the new sterile connector assembly 1201 may resume.

Accordingly, the present disclosure provides features of coupling devices that can provide for efficient and safe maintenance of fluid connections, such as the connections used for transferring medical fluids toward or away from a patient. Further, the coupling devices of the present disclosure can maintain the fluid connection by resisting unintended disconnection when a pulling or tension force is applied to the coupling device, while also permitting disconnection of the fluid connection when a pulling or tension force exceeds a threshold, thereby preventing injury to a patient or a caregiver. Additionally, the coupling devices of the present disclosure permit efficient and safe reconnection of the fluid line by permitting the coupling device to be reconnected to the fluid transfer device, thereby reestablishing the fluid pathway without requiring replacement of the coupling device.

Accordingly, the coupling device 100 can provide for efficient and safe maintenance of fluid connections, can maintain the fluid connection of a medical tubing, and can permit disconnection and reconnection of the fluid pathway with a patient.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 11, clause 17, clause, clause 23, clause 26, or clause 29. The other clauses can be presented in a similar manner.

Clause 1. A fluid connector assembly, comprising: a first connector having a first housing, a second connector configured to couple with the first connector, the second connector having a second housing, a connecting mechanism disposed on at least one of the first connector and the second connector for removably coupling the first connector and the second connector, wherein, when a predetermined pull force is applied to at least one of the first connector and the second connector, the connecting mechanism disengages thereby allowing the first connector and the second connector to be decoupled, and wherein, when the first connector and the second connector are decoupled, an interior surface of one of the first connector is exposed thereby allowing for disinfection.

Clause 2. The fluid connector assembly described above, wherein the connecting mechanism comprises a first connecting member coupled to one of the first connector or the second connector.

Clause 3. The fluid connector assembly described above, wherein the first connecting member comprises a projection member disposed at a first end of the first connecting member.

Clause 4. The fluid connector assembly described above, further comprising a second connecting member, and wherein the first connecting member is coupled to the first connector and the second connecting member is coupled to the second connector.

Clause 5. The fluid connector assembly described above, wherein the second connecting member comprises a groove for receiving the projection member, wherein, when the first connector and the second connector are coupled, the first connecting member and the second connecting member are coupled such that the projection is received within the groove, and wherein, when the predetermined force is applied, the first connecting member and the second connecting member are decoupled.

Clause 6. The fluid connector assembly described above, wherein the first connecting member is a press ring, and wherein the first connecting member is flexible.

Clause 7. The fluid connector assembly described above, wherein the second connecting member further comprises a groove for receiving the projection of the press ring.

Clause 8. The fluid connector assembly described above, wherein, when the first connector and the second connector are coupled, the first connecting member and the second connecting member are coupled, and wherein, when the predetermined force is applied, the first connecting member and the second connecting member are decoupled.

Clause 9. The fluid connector assembly described above, wherein the projection is configured to engage an elastomer ring, and wherein, when the predetermined force is applied, the projection disengage the elastomer ring thereby decoupling the first connector and the second connector.

Clause 10. The fluid connector assembly described above, wherein the connecting member comprises an elastic band and one or more threads disposed around the first connector or second connector, and wherein, the elastic band is disposed around the one or more threads.

Clause 11. The fluid connector assembly described above, wherein, the elastic band is configured to deflect until the predetermined threshold force is applied, and wherein, when the predetermined threshold force is applied, the elastic band expands allowing the one or more threads to pull thereby decoupling the first connector and the second connector.

Clause 12. A fluid connector assembly comprising: a first connector having a first housing; a second connector configured to couple with the first connector, the second connector having a second housing; a connecting mechanism disposed on at least one of the first connector and the second connector for removably coupling the first connector and the second connector, and wherein, when a pull force of a predetermined threshold is applied to one of the first connector and the second connector, the first connector and the second connector are decoupled.

Clause 13. The fluid connector assembly described above, wherein the connecting mechanism comprises a first connecting member coupled to one of the first connector or the second connector.

Clause 14. The fluid connector assembly described above, wherein the first connecting member comprises a projection member disposed at a first end of the first connecting member.

Clause 15. The fluid connector assembly described above, further comprising a second connecting member, and wherein the first connecting member is coupled to the first connector and the second connecting member is coupled to the second connector.

Clause 16. The fluid connector assembly described above, wherein the second connecting member comprises a groove for receiving the projection member, wherein, when the first connector and the second connector are coupled, the first connecting member and the second connecting member are coupled such that the projection is received within the groove, and wherein, when the predetermined force is applied, the first connecting member and the second connecting member are decoupled.

Clause 17. The fluid connector assembly described above, wherein the first connecting member is a press ring, and wherein the first connecting member is flexible.

Clause 18. The fluid connector assembly described above, wherein the second connecting member further comprises a groove for receiving the projection of the press ring.

Clause 19. The fluid connector assembly described above, wherein, when the first connector and the second connector are coupled, the first connecting member and the second connecting member are coupled, and wherein, when the predetermined force is applied, the first connecting member and the second connecting member are decoupled.

Clause 20. The fluid connector assembly described above, wherein the projection is configured to engage an elastomer ring, and wherein, when the predetermined force is applied, the projection disengage the elastomer ring thereby decoupling the first connector and the second connector.

Clause 21. The fluid connector assembly described above, wherein the connecting member comprises an elastic band and one or more threads disposed around the first connector or second connector, and wherein, the elastic band is disposed around the one or more threads.

Clause 22. The fluid connector assembly described above, wherein, the elastic band is configured to deflect until the predetermined threshold force is applied, and wherein, when the predetermined threshold force is applied, the elastic band expands allowing the one or more threads to pull thereby decoupling the first connector and the second connector.

Clause 23. A fluid connector assembly comprising: a first connector having a first housing; a second connector configured to couple with the first connector, the second connector having a first connector member and a second connector member; a connecting mechanism disposed on at least one of the first connector and the second connector for removably coupling the first connector and the second connector, wherein, when a pull force of a predetermined threshold is applied to one of the first connector and the second connector, the first connector and the second connector are decoupled, and wherein, when the first connector and the second connector are decoupled, the first connector member can be decoupled from the second connector member.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector assembly, comprising:
a first connector having a first housing,
a second connector configured to couple with the first connector, the second connector having a second housing,
a connecting mechanism disposed on at least one of the first connector and the second connector for removably coupling the first connector and the second connector,
wherein, when a predetermined pull force is applied to at least one of the first connector and the second connector, the connecting mechanism disengages thereby allowing the first connector and the second connector to be decoupled, and
wherein, when the first connector and the second connector are decoupled, an interior surface of one of the first connector is exposed thereby allowing for disinfection.

2. The fluid connector assembly of claim 1, wherein the connecting mechanism comprises a first connecting member coupled to one of the first connector or the second connector.

3. The fluid connector assembly of claim 2, wherein the first connecting member comprises a projection member disposed at a first end of the first connecting member.

4. The fluid connector assembly of claim 2, further comprising a second connecting member, and
wherein the first connecting member is coupled to the first connector and the second connecting member is coupled to the second connector.

5. The fluid connector assembly of claim 4, wherein the second connecting member comprises a groove for receiving the projection member,
wherein, when the first connector and the second connector are coupled, the first connecting member and the second connecting member are coupled such that the projection is received within the groove, and
wherein, when the predetermined force is applied, the first connecting member and the second connecting member are decoupled.

6. The fluid connector assembly of claim 4, wherein the first connecting member is a press ring, and
wherein the first connecting member is flexible.

7. The fluid connector assembly of claim 6, wherein the second connecting member further comprises a groove for receiving the projection of the press ring.

8. The fluid connector assembly of claim 4, wherein, when the first connector and the second connector are coupled, the first connecting member and the second connecting member are coupled, and
wherein, when the predetermined force is applied, the first connecting member and the second connecting member are decoupled.

9. The fluid connector assembly of claim 3, wherein the projection is configured to engage an elastomer ring, and
wherein, when the predetermined force is applied, the projection disengage the elastomer ring thereby decoupling the first connector and the second connector.

10. The fluid connector assembly of claim 1, wherein the connecting member comprises an elastic band and one or more threads disposed around the first connector or second connector, and
wherein, the elastic band is disposed around the one or more threads.

11. The fluid connector assembly of claim 10, wherein, the elastic band is configured to deflect until the predetermined threshold force is applied, and
wherein, when the predetermined threshold force is applied, the elastic band expands allowing the one or more threads to pull thereby decoupling the first connector and the second connector.

12. A fluid connector assembly comprising:
a first connector having a first housing;
a second connector configured to couple with the first connector, the second connector having a second housing;
a connecting mechanism disposed on at least one of the first connector and the second connector for removably coupling the first connector and the second connector, and
wherein, when a pull force of a predetermined threshold is applied to one of the first connector and the second connector, the first connector and the second connector are decoupled.

13. The fluid connector assembly of claim 12, wherein the connecting mechanism comprises a first connecting member coupled to one of the first connector or the second connector.

14. The fluid connector assembly of claim 13, wherein the first connecting member comprises a projection member disposed at a first end of the first connecting member.

15. The fluid connector assembly of claim 13, further comprising a second connecting member, and
wherein the first connecting member is coupled to the first connector and the second connecting member is coupled to the second connector.

16. The fluid connector assembly of claim 15, wherein the second connecting member comprises a groove for receiving the projection member,
wherein, when the first connector and the second connector are coupled, the first connecting member and the second connecting member are coupled such that the projection is received within the groove, and
wherein, when the predetermined force is applied, the first connecting member and the second connecting member are decoupled.

17. The fluid connector assembly of claim 15, wherein the first connecting member is a press ring, and
wherein the first connecting member is flexible.

18. The fluid connector assembly of claim 17, wherein the second connecting member further comprises a groove for receiving the projection of the press ring.

19. The fluid connector assembly of claim 15, wherein, when the first connector and the second connector are coupled, the first connecting member and the second connecting member are coupled, and
wherein, when the predetermined force is applied, the first connecting member and the second connecting member are decoupled.

20. The fluid connector assembly of claim 14, wherein the projection is configured to engage an elastomer ring, and
wherein, when the predetermined force is applied, the projection disengage the elastomer ring thereby decoupling the first connector and the second connector.
